(19) United States
(12) Patent Application Publication
(10) Pub. No.: US 2008/0071117 A1
(43) Pub. Date: Mar. 20, 2008

(54) Method for the Continuous Production of Dmc Catalysts

(75) Inventors: Edward Bohres, Mannheim (DE); Michael Triller, Mannheim (DE); Bernd Bechtloff, Ludwigshafen (DE); Raimund Ruppel, Dresden (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/576,105

(22) PCT Filed: Sep. 28, 2005
(86) PCT No.: PCT/EP05/10492

§ 371 Date: Mar. 27, 2007

(30) Foreign Application Priority Data
Oct. 5, 2004 (DE) 102004048735.9

(51) Int. Cl. C07C 41/01

(52) U.S. Cl. 568/679

(57) ABSTRACT

The invention provides a continuous process for preparing DMC catalysts, which comprises continuously feeding the solutions of a metal salt and of a hexacyanometallate compound and, if appropriate, organic ligands and/or organic additives into a continuous reactor, and continuously withdrawing the resulting suspension of the DMC compound from the reactor.

The invention provides a process for continuously preparing multimetal cyanide compounds which may be used as catalysts for the addition of alkylene oxides to H-functional compounds. These compounds are frequently also referred to as DMC compounds or DMC catalysts.

The use of DMC catalysts for preparing polyether alcohols by adding alkylene oxides to H-functional compounds has been known for some time. The resulting polyether alcohols may be used as surfactants, as carrier oils, but mainly as starting materials for preparing polyurethanes. Compared to basic catalysts, DMC catalysts lead to products having a lower content of unsaturated fractions in the polyether chain. In addition, the alkylene oxides are added at a higher rate.

A multitude of documents on DMC catalysts, their preparation, crystal structure and use for the preparation of polyurethanes is known.

The DMC catalysts are prepared typically by combining the solutions of a metal salt and of a hexacyanometallate compound, and subsequently removing purifying and, if appropriate, drying the resulting multimetal cyanide compound. Typically, the DMC catalysts are prepared in the presence of ligands and/or surfactants.

Such processes are described, for example, in U.S. Pat. No. 3,278,458, EP 862 997 and DD 203 734.

Since the preparation of DMC catalysts is costly and inconvenient, there has been no shortage of attempts in the past to simplify the preparation. For instance, U.S. Pat. No. 5,891,818 describes a process for preparing DMC catalysts by combining a metal salt solution with the solution of a hexacyanometallate compound, in which a portion of the reaction mixture is removed and recycled into the reactor as spray via a nozzle. This procedure is intended to suppress the foam formation in the reactor and bring about better mixing of the reaction mixture. The circuit includes an inline mixer, by which the catalyst particles are further comminuted owing to shear forces, which leads to a higher activity of the catalyst. However, this procedure is still costly and inconvenient, and the nozzle can become blocked by the catalyst particles.

WO 01/39883 describes a process for preparing DMC catalysts, in which a metal salt solution is combined with the solution of a hexacyanometallate compound in a mixer nozzle. A disadvantage in this process is that particles can form actually within the nozzle, which leads to a pressure drop in the nozzle up to and including blockages.

A disadvantage of all batch processes for preparing DMC catalysts is also that the product parameters of the individual batches can be different.

It is an object of the present invention to develop a process for preparing DMC catalysts, in which DMC catalysts can be prepared in a simple and operationally reliable manner, with uniform quality and high space-time yield. At the same time, there should be no deterioration in the catalytic activity of the DMC catalysts.

This object is achieved, surprisingly, by continuously metering the reactants used to prepare the DMC catalysts into a continuous reactor and continuously withdrawing the resulting DMC catalyst from the reactor.

The invention thus provides a continuous process for preparing DMC catalysts, which comprises continuously feeding the solutions of a metal salt and of a hexacyanometallate compound and, if appropriate, organic ligands and/or organic additives into a continuous reactor, and continuously withdrawing the resulting suspension of the DMC compound from the reactor.

The present invention further provides the DMC catalysts prepared by the process according to the invention and for the use thereof to prepare polyether alcohols.

The continuous reactors used may be tubular reactors and preferably continuous stirred tank reactors.

The solutions of the metal salt and of the hexacyanometallate compound, also referred to hereinbelow as reactant solutions, can be added to the reactor, especially the continuous stirred tank, through a mixer nozzle or through inlet tubes at the surface of the reaction mixture or immersed. The use of mixer nozzles to premix the reactant solutions is not necessary. There is thus no risk of blockage of the nozzles, which leads to uniform and disruption-free operation of the reactor.

The resulting DMC catalyst suspension is withdrawn continuously from the reactor. In the case of the use of a continuous stirred tank as the reactor, this may be ensured, for example, by virtue of a fill level control coupled with a bottom valve, with a continuous drawoff via a pump or with an overflow.

When a continuous stirred tank is used, there is preferably an energy input through the stirrer in the range between 10⁻²-10 kW/M³. The average residence time in the reactor is preferably in the range between 1 and 180 minutes. The temperature in the reactor is preferably between 10 and 80° C., more preferably between 15 and 60° C., in particular between 20 and 50° C.

When these conditions are maintained, DMC catalysts having a high catalytic activity are obtained.

An apparatus for comminuting the particles formed may be attached to the outlet of the suspension from the reactor. To this end, for example, a wet rotor mill may be used. This leads to a more uniform distribution of the particle size in the suspension.

After the withdrawal from the reactor, the suspension of the DMC compound is typically sent to a washing, filtration, redispersion and, if appropriate, a drying step. These workup steps may likewise be operated continuously. However, it is also possible to collect the suspension in intermediate vessels and send it batchwise to the workup steps mentioned.

The washing may be effected either only with water, with an organic ligand or any mixtures of the two.

It is also possible in principle to dispense with the drying of the DMC catalysts and to use them in the redispersed form as a suspension to prepare the polyether alcohols. It is equally possible to suspend the DMC catalyst after the drying and to use it in this form to prepare the polyether alcohols.

If drying of the DMC catalysts is carried out, this is effected preferably at a temperature in the range between 20 and 150° C., in particular between 30 and 100° C., and a pressure between 0.01 bar and 1 bar, in particular between 0.05 bar and 0.7 bar.

DMC catalysts prepared by the process according to the invention may, depending on the reactants and assistants used and the preparation conditions, have a different crystal structure. Thus, the DMC catalysts may have a crystalline or an amorphous structure. Crystalline DMC catalysts are described, for example, in WO 99116775; amorphous DMC catalysts are described, for example, in EP 654 302. The catalysts may also be semicrystalline, which means that they comprise both crystalline and amorphous fractions.

Among the crystalline DMC catalysts, particular preference is given to those having a monoclinic crystal structure.

In a further preferred embodiment, the DMC catalysts prepared by the process according to the invention have a platelet shape, as described, for example, in WO 00174845.

The DMC catalysts prepared by the process according to the invention usually have the general formula (I)
M¹ₐ[M²(CN)_{b}(A)_{c}]_{d}·fM¹_{g}Xₙ·h(H₂O)eL·kP (I)
where

M¹ is a metal ion selected from the group comprising Zn²⁺, Fe²⁺, Fe³⁺, Co²⁺, Co³⁺, Ni²⁺, Mn²⁺, Sn²⁺, Sn⁴⁺, Pb²⁺, Mo⁴⁺, Mo⁶⁺, Al³⁺, V⁴⁺, V⁵⁺, Sr²⁺, W⁴⁺, W⁶⁺, Cr²⁺, Cr³⁺, Cd²⁺, Cu²⁺, La³⁺, Ce³⁺, Ce⁴⁺, Eu³⁺, Mg²⁺, Ti³⁺, Ti⁴⁺, Ag⁺, Rh²⁺, Ru²⁺, Ru³⁺, Pd²⁺

M² a metal ion selected from the group comprising Fe²⁺, Fe³⁺, Co²⁺, Co³⁺, Mn²⁺, Mn³⁺, Ni²⁺V⁴⁺, V⁵⁺, Cr²⁺, Cr³⁺, Rh³⁺, Ru²⁺, Ir³⁺

and M¹ and M² are different,

A is an anion selected from the group comprising halide, hydroxide, sulfate, hydrogensulfate, carbonate, hydrogencarbonate, cyanide, thiocyanate, isocyanate, cyanate, carboxylate, oxalate, nitrate, nitrosyl, phosphate, hydrogenphosphate or dihydrogenphosphate

X is an anion selected from the group comprising halide, hydroxide, sulfate, hydrogensulfate, carbonate, hydrogencarbonate, cyanide, thiocyanate, isocyanate, cyanate, carboxylate, oxalate, nitrate or nitrite (NO₂⁻), and the uncharged species CO, H₂O and NO,

L is a water-miscible ligand selected from the group comprising alcohols, aldehydes, ketones, ethers, polyethers, esters, polyesters, polycarbonate, ureas, amides, nitriles and sulfides or mixtures thereof,

P is an organic additive selected from the group comprising polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylamide-co-maleic acid), polyacrylonitrile, polyaikyl acrylates, polyalkyl methacrylates, polyvinyl methyl ethers, polyvinyl ethyl ethers, polyvinyl acetate, polyvinyl alcohol, poly-N-vinylpyrrolidone, poly(N-vinylpyrrolidone-co-acrylic acid), polyvinyl methyl ketone, poly(4-vinylphenol), poly(acrylic acid-co-styrene), oxazoline polymers, polyalkyleneimines, maleic acid and maleic anhydride copolymer, hydroxyethylcellulose, polyacetates, ionic surface- and interface-active compounds, gallic acid and salts, esters or amides thereof, carboxylic esters of polyhydric alcohols and glycosides,

and

a, b, d, q and n are integers or fractions greater than zero,

c, f, e, h and k are integers or fractions greater than or equal to zero,

where

a, b, c and d, and also q and n, are selected such that electrical neutrality is ensured.

These catalysts may, as described, be crystalline or amorphous. In the case that k is zero, preference is given to crystalline double metal cyanide compounds. In the case that k is greater than zero, preference is given either to crystalline, semicrystalline or substantially amorphous catalysts.

Preferred embodiments of the DMC catalysts of the general formula (I) prepared by the process according to the invention are those in which k is greater than zero. This DMC catalyst comprises at least one multimetal cyanide compound, at least one organic ligand and at least one organic additive P.

In another preferred embodiment, k is zero, e is optionally also zero and X is exclusively carboxylate, preferably formate, acetate and propionate. In this embodiment, which is described, for example, in WO 99/16775, preference is given to crystalline double metal cyanide catalysts.

Preferred examples of M¹ are Zn²⁺, Fe²⁺, Co²⁺, Fe³⁺, Mn²⁺. Preferred examples of M² are Fe²⁺, Fe³⁺, Co²⁺, Co³⁺, Ir³⁺. Preferred examples of A are halide and carboxylate, especially acetate.

Together with or instead of organic ligands, it is possible in the preparation of the DMC catalysts to use at least one surfactant. This surfactant is not incorporated into the catalyst and is removed from the catalyst virtually fully by the washing of the catalyst. The thus prepared DMC catalysts have improved morphology.

In a further embodiment of the DMC catalysts prepared by the process according to the invention, as described in WO 01/03830, organic sulfones of the general form R—S(O)₂—R or sulfoxides of the general form R—S(O)—R are used as an organic complexing agent L. The advantages of this embodiment are short induction times and moderate exothermicity in the preparation of the polyether alcohols.

In a preferred embodiment of the process according to the invention, the reaction is carried out at a pH>1, preferably>4, more preferably>7. Under these conditions, crystalline DMC catalysts having a monoclinic crystal structure are formed.

In another preferred embodiment of the catalysts, f, e and k are not equal to zero. These catalysts are DMC catalysts which comprise a water-miscible organic ligand, preferably in amounts of from 0.5 to 30% by weight, and an organic additive, preferably in amounts of from 5 to 80% by weight. Such catalysts are described, for example, in WO 98/06312.

The catalysts may be prepared when a stirred tank is used with vigorous stirring, for example with a Turrax®, as described, for example, in U.S. Pat. No. 5,158,922.

The DMC compounds prepared by the process according to the invention are usually, as described, used as catalysts for adding alkylene oxides to H-functional starter substances. The thus obtained products may be used as surfactants, carrier oils or as polyether alcohols for the preparation of polyurethanes.

The alkylene oxides used may be all known alkylene oxides, for example ethylene oxide, propylene oxide, butylene oxide, styrene oxide. In particular, the alkylene oxides used are ethylene oxide, propylene oxide and mixtures of the compounds mentioned.

To prepare polyether alcohols for use as raw materials for polyurethane preparation, the starter substances used are in particular polyfunctional alcohols, and the alkylene oxides used are preferably ethylene oxide and/or propylene oxide.

The starter substances used are H-functional compounds. In particular, alcohols having a functionality of from 1 to 8, preferably from 2 to 8, are used. To prepare polyether alcohols which are used for flexible polyurethane foams, the starter substances used are preferably alcohols having a functionality of from 2 to 4, in particular of 2 and 3. Examples are ethylene glycol, propylene glycol, glycerol, trimethylolpropane, pentaerythritol. When the alkylene oxides are added on by means of DMC catalysts, it is advantageous, together with or instead of the alcohols mentioned, to use reaction products thereof with alkylene oxides, especially propylene oxide. Such compounds preferably have a molar mass up to 500 g/mol. In the preparation of these reaction products, the alkylene oxides can be added on with any catalysts, for example with basic catalysts. The polyether alcohols for the preparation of flexible polyurethane foams usually have a hydroxyl number in the range between 20 and 100 mg KOH/g.

In the preparation of the polyether alcohols used for the process according to the invention, the alkylene oxides can be added on by the known processes. For instance, it is possible that the polyether alcohols contain only one alkylene oxide. When a plurality of alkylene oxides is used, what is known as a blockwise arrangement, in which the alkylene oxides are added individually and successively, or what is known as a random arrangement, in which the alkylene oxides are metered in together, are possible. It is also possible to incorporate both blockwise and random sections into the polyether chain when the polyether alcohols are prepared.

The alkylene oxides are added on under the conditions customary therefor, such as temperatures in the range from 60 to 180° C., preferably between 90 and 140° C., in particular between 100 to 130° C., and pressures in the range from 0 to 20 bar, preferably in the range from 0 to 10 bar and in particular in the range from 0 to 5 bar. Before the start of the alkoxylation, the mixture of starter substance and DMC catalyst may be pretreated by stripping according to the teaching of WO 98/52689.

On completion of the adding-on of the alkylene oxides, the polyether alcohol is usually worked up by customary processes, by removing the unconverted alkylene oxides and volatile constituents, typically by distillation, steam or gas stripping, and/or other methods of deodorization. If required, a filtration may be effected.

On completion of the adding-on of the alkylene oxides, the catalyst may be removed from the reaction mixture. However, it is possible for most uses of the polyether alcohols, especially in the preparation of polyurethanes, to leave it in the product.

In a particular embodiment, the polyether alcohols can also be prepared continuously. Such a procedure is described, for example, in WO 98103571 or in JP H6-16806. In this procedure, alkylene oxides and starter substance are metered continuously into a continuous reactor, and the resulting polyether alcohol is withdrawn continuously.

The polyether alcohols prepared using DMC catalysts are, as detailed, usually used to prepare flexible polyurethane foams by reaction with polyisocyanates.

The properties of the DMC catalysts prepared by the process according to the invention do not have any disadvantages compared to other catalysts prepared by the customary batchwise process. The process according to the invention allows the level of complexity and expense in the preparation of DMC catalysts to be distinctly lowered. In addition, the DMC catalysts prepared by the process according to the invention have uniform properties.

The invention will be illustrated in detail by the examples which follow.

### EXAMPLE 1 (COMPARATIVE)

### Semicontinuous Preparation of a DMC Catalyst Suspension

Solution 1 consisted of an aqueous zinc acetate solution (2.6% zinc), solution 2 of an aqueous potassium hexacyanocobaltate solution with 0.9% cobalt. Solution 1 was metered at 7.91 kg/h and solution 2 at 10 kg/h via a mixer nozzle into a 3-liter stirred vessel. Both solutions contained 2% by weight of a surfactant (Pluronic® PE6200 from BASF AG). After the stirred tank had been charged, the feed was stopped and the stirring of the DMC suspension present was discharged continued through a bottom outlet at a temperature of 20° C. in the stirred vessel and an energy input through stirring of 1 W/I for 1 h. Subsequently, the catalyst was filtered off, washed with water and dried at 60° C.

### EXAMPLE 2

Continuous preparation of a DMC catalyst suspension using a mixer nozzle

Solution 1 consisted of an aqueous zinc acetate solution (2.6% zinc), solution 2 of an aqueous potassium hexacyanocobaltate solution with 0.9% cobalt. Solution 1 was metered continuously at 7.91 kg/h and solution 2 at 10 kg/h via a mixer nozzle into a 3-liter stirred vessel. Both solutions contained 2% by weight of a surfactant (Pluronic® PE6200 from BASF AG). After the stirred tank had been charged, metered addition was continued; the DMC suspension present was discharged continuously through a bottom outlet valve under fill level control at a temperature of 20° C. in the stirred vessel and an energy input through stirring of 1 W/I. The average residence time in the stirred tank was 10 min. To ensure attainment of the steady state, the experiment was carried out over 10 average residence times. Subsequently, the catalyst was filtered off, washed with water and dried at 60° C.

### EXAMPLE 3

### Continuous Preparation of a DMC Catalyst Suspension Without Mixer Nozzle

Solution 1 consisted of an aqueous zinc acetate solution (2.6% zinc), solution 2 of an aqueous potassium hexacyanocobaltate solution with 0.9% cobalt. Solution 1 was metered continuously at 3.95 kg/h and solution 2 at 5 kg/h via inlet tubes into a 3-liter stirred vessel. Both solutions contained 2% by weight of a surfactant (Pluronic® PE6200 from BASF AG). After the stirred tank had been charged, metered addition was continued; the DMC suspension present was discharged continuously through a bottom outlet valve under fill level control at a temperature of 35° C. in the stirred vessel and an energy input through stirring of 1 W/I. The average residence time in the stirred tank was 20 min. To ensure attainment of the steady state, the experiment was carried out over 10 average residence times. Subsequently, the catalyst was filtered off, washed with water and dried at 60° C.

### Method for Determining the Activity of the Catalysts

The specified amounts of the DMC catalyst to be tested were added to 10 g of a glycerol propoxide having a molecular weight Mw of 1000 g/mol, referred to hereinbelow as VP900, and the mixture was dispersed to give a concentrate with an Ultra-Turrax® T25 dispersion unit from IKA for 5 minutes. Afterward, a further 120 g of VP900 were added and homogenization was once again effected with the Ultra-Turrax® T25 for 5 minutes. Afterward, this VP900/DMC mixture was kept in a stirred autoclave at 100° C. at 3 mbar for 2 hours. Subsequently, 70 g of propylene oxide were metered in all at once at 130° C. From the rise of temperature and pressure, the maxima were recorded and registered as the initiation time and simultaneously rating for the activity. After the propylene oxide had fully reacted, recognizable by the pressure falling to a constant level, the polyether alcohol, after inertization with nitrogen, was discharged from the autoclave.

Results:

| Catalyst | Concentration [ppm] | Initiation time [min] | pₘₐₓ/Tₘₐₓ |
|---|---|---|---|
| Example 1 | 100 | 8 | 8.6 bar/165° C. |
| Example 2 | 100 | 7 | 8.9 bar/175° C. |
| Example 3 | 100 | 10 | 8.4 bar/169° C. |
| Comparison* | 100 | 8 | 8.6 bar/172° C. |

*EP862 997, Example 1

As is evident, the catalytic activity of the DMC catalysts prepared by the process according to the invention is comparable with that of DMC catalysts from conventional processes.

1-14. (canceled)

15. A continuous process for preparing DMC catalysts, which comprises continuously feeding the solutions of a metal salt and of a hexacyanometallate compound and, if appropriate, organic ligands and/or organic additives into a continuous stirred tank reactor, and continuously withdrawing the resulting suspension of the DMC compound from the reactor.

16. The process according to claim 15, wherein the starting compounds are fed in through inlet tubes.

17. The process according to claim 15, wherein the inlet tubes are mounted at the surface of the reaction mixture in the reactor or immersed.

18. The process according to claim 15, wherein the starting compounds are fed in via a mixer nozzle.

19. The process according to claim 15, wherein the suspension is withdrawn continuously from the reactor by virtue of a fill level control coupled with a bottom valve, with a drawoff via a pump or with an overflow.

20. The process according to claim 15, wherein an apparatus for comminuting the particles formed is attached to the withdrawal point from the continuous reactor.

21. The process according to claim 15, wherein the reaction is carried out at a temperature of 10-80° C.

22. The process according to claim 15, wherein the average residence time in the reactor is in the range between 1 and 180 minutes.

23. The process according to claim 15, wherein the energy input into the continuous reactors is 10⁻²−10 kW/m³.

24. A DMC catalyst preparable according to claim 15.

25. The DMC catalyst according to claim 24, which is crystalline.

26. The DMC catalyst according to claim 24, which has a monoclinic crystal structure.

27. The process for adding alkylene oxides to compounds having active hydrogen atoms, which comprises using DMC catalysts according to claim 24.

28. A process for preparing polyether alcohols by adding alkylene oxides to compounds having at least two hydrogen atoms reactive with isocyanates using catalysts, which comprises using, as catalysts, DMC catalysts according to claim 24.
